(19) United States
(12) Patent Application Publication
(10) Pub. No.: US 2007/0259037 A1
(43) Pub. Date: Nov. 8, 2007

(54) Method and composition for treatment of inflammatory conditions

(76) Inventors: Pol-Henri Guivarc'h, Montreal (CA); Gary Robinson, Montreal (CA)

Correspondence Address: NIXON & VANDERHYE P.C., 8th Floor, 1100 North Glebe Rd., Arlington, VA, 22201-4714, US

(21) Appl. No.: 11/824,830

(22) Filed: Jul. 2, 2007

Related U.S. Application Data
(63) Continuation of application No. 10/126,980, filed on Apr. 22, 2002, now abandoned.
Application No. 10/126,980 is a continuation of application No. 09/245,912, filed on Feb. 8, 1999, now abandoned.
Application No. 09/245,912 is a continuation-in-part of application No. 09/022,008, filed on Feb. 11, 1998, now abandoned.

(51) Int. Cl. A61K 31/58; A61K 31/00; A61K 9/48; A61P 29/00

(52) U.S. Cl. 424/463; 514/1; 514/174

(57) ABSTRACT

Methods of treating inflammation of the gastrointestinal tract and/or systemic or local inflammation by administering a steroid anti-inflammatory or a non-steroid anti-inflammatory drug in conjunction with polyunsaturated fatty acids or their derivatives and optionally also a pharmacologically active antioxidant and compositions for practicing these methods are described.

This application is a continuation-in-part of application Ser. No. 09/022,008, filed Feb. 11, 1998.

This invention provides compositions for preventing, mitigating, or treating inflammation of the gastrointestinal tract and systemic or local symptoms of inflammation, which contain an anti-inflammatory drug, in combination with a source of polyunsaturated fatty acids, such as omega-3, omega-6, and omega-9 polyunsaturated fatty acids, and optionally also a source of a pharmacologically active antioxidant such as tocopherols (e.g., alpha- and/or gamma-tocopherol). Also described are methods for preventing, mitigating, or treating such inflammation, by means of orally, locally or otherwise administering compositions of this invention to a mammal.

### BACKGROUND OF THE INVENTION

The present invention relates to a method and a composition treating inflammatory conditions of “the gastrointestinal tract” (GIT); for those conditions that may be distant from, but sequelae of, GIT; and for systemic or localized (i.e., non-intestinal) inflammations not related to GIT inflammation.

Inflammation (defined below) can be chronic or acute, or can alternate between the two states. Inflammation of the GIT may be due to etiologies as diverse as infection, reaction to drugs or other foreign (irritating) substances, or to diseases such as Inflammatory Bowel Diseases (Crohn's disease; ulcerative colitis). The primary focus of this invention is IBD, but is intended only as an example and not as a restriction or limitation to that disease, site or form of inflammation.

As an example of a disease addressed by the present invention, IBD is characterized by periods of varying disease activity, i.e., quiescent, intermediate, and acute (active) phases. Depending on the phase symptoms can range from none, to mild and somewhat tolerable, to severe and requiring hospitalization for treatment. The etiology of the disease is unknown, but the principal pathophysiology seems to be the result of a “hyper-inflammatory” condition within the GIT. During the intermediate and acute phases, extra-intestinal (systemic) involvement may occur. e.g., ophthalmic, arthritic. The systemic conditions themselves are inflammatory in pathophysiology, and present discrete management approaches.

Treatment of chronic and acute aspects of IBD include, but is not limited to, drug therapy. During the quiescent phases, i.e., during remission, some patients require no medical treatment, although dietary management is often instituted as adjunctive therapy (see below). In the intermediate stages, patients experience mild symptoms which can be rendered tolerable with low (maintenance) doses of various medications. Drugs employed include both steroidal and non-steroidal anti-inflammatory drugs (e.g., steroids, such as prednisone and prednisolone), as well as derivatives of 5-aminosalicylic acid e.g., Mesalamine, Sulphasalazine, Olsalazine, and Balsalazide), and other drugs known collectively as “immunosuppressives” (e.g., Cyclosporine. Azathioprine, and 6-Mercaptopurine). Finally, adjunctive diet therapy is usually instituted.

For active stages of Inflammatory Bowel Disease, for example in Crohn's disease, intensive drug therapy is indicated, sometimes in a hospital setting. Steroids and immunosuppressive drugs are employed, in higher doses than in chronic phases. A new steroid, budesonide (Astra) has recently been introduced. Rutgeerts (1994) have shown budesonide to be nearly as effective, and with fewer side-effects compared to prednisone, in the acute phase of Crohn's Disease (CD). Budesonide is especially useful because it is delivered “topically”, i.e., to the luminal side of the GIT, which in CD is the site of inflammation; this reduces undesirably high concentrations of the drug in the systemic circulation, which can lead to adverse manifestations of the drug. Further, the provision of budesonide, or other topically active drugs, in a sustained release formulation would be a preferred treatment.

Adjunctive therapy usually includes dietary modifications, which are variable in type and extent. For patients in remission, medical treatment is not employed. However, some efforts have been made to delay the onset of relapse to the active states. Such efforts often employ dietary treatment. For example, patients may limit themselves to diets low in residue (fiber) and “bland” diets. A recent study demonstrated that patients who consumed a source of omega-3 polyunsaturated fatty acids daily for one year suffered fewer relapses than those taking placebo oil. For those with intermediate symptoms, low-dose medications may be combined with dietary restrictions similar to those noted above.

Some patients have such severe acute phases that total bowel rest is employed, wherein nutrition is provided in the form of total parenteral nutrition (TPN). Others may do well—perhaps as well as those on drugs—with special diets known as “medical foods”. Medical foods are usually liquid, are defined formulas, are intended for dietary management of specific diseases, and are administered under the supervision of a physician. Since the chronic phases of the disease often lead to partial anorexia, and may include malabsorption, patients not uncommonly manifest degrees of malnutrition. Such malnutrition can include deficiencies in essential fatty acids (EFA), and dietary therapy often includes good sources of EFA. Once stabilized, patients may be placed on restricted diets (low in residue; “bland”). Only when remission is complete are “normal” diets introduced.

The “therapeutic” (i.e., induction of remission) properties of some medical foods has led to speculation as to the identity and the mechanism of action of the responsible dietary components. Many food components of such diets have received attention in this regard, especially the lipid components known as “omega-3 polyunsaturated fatty acids” (“omega-3”) and derivatives such as triglycerides and esters, which themselves have anti-inflammatory metabolic properties. In fact, omega-3 have been shown to have anti-inflammatory effects in clinical trials of asthma, rheumatoid arthritis, and cancer. A recent clinical trial showed that omega-3 slowed the rate of relapse in patients with “quiet” Crohn's disease (Belluzzi, et al. 1996). This is consistent with an anti-inflammatory effect of omega-3. Other clinical studies have shown benefit of omega-3 supplementation in intestinal diseases (Stenson et al., 1992, Mate et al., 1991; and other references in Belluzzi et al., 1996). Further still, other studies using lipid sources rich in omega-6 and omega-9 polyunsaturated fatty acids have also suggested that patients suffering from inflammatory conditions can gain symptomatic relief from appropriate dietary supplementation. Whilst the primary focus of this invention is the combination of anti-inflammatory drugs with refined edible oils enriched with omega-3 polyunsaturated fatty acids, other sources of enriched polyunsaturated fatty acids, such as omega-6 and omega-9 polyunsaturated fatty acids, can substitute in formulations for omega-3. The use of omega-3 throughout this invention is intended only as an example and not as a restriction or limitation to the specifications of drug and oil formulations.

The addition of antioxidants to the invention can provide both clinical and formulation benefits. As an example, but not a limitation to our invention, the nutrient known categorically as “vitamin E” consists of a mixture of isomers of tocopherol, of which the alpha and gamma forms are the most common in blood and in most foods. Tocopherols are potent antioxidants in vitro, and are usually added to preparations of polyunsaturated fatty acids to minimize oxidation. Tocopherols also exert beneficial effects in vivo, in animals and man, due possibly to their antioxidant properties. We are not aware that tocopherols have been studied with regard to GIT inflammation, their beneficial clinical effects give a second reason for adding them, beyond the issue of formulation stability. It was recently shown that the alpha and gamma isomers of the tocopherols differ in their specificity of antioxidant capacity, that is, a combination of the two forms would theoretically give better overall protection against excessive oxidation.

A combination of drug and the nutrients, omega-3, and of tocopherols, may improve the efficacy of treatment in IBD and other inflammatory conditions, in greater than additive fashion, which is the subject of the present invention.

### SUMMARY OF THE INVENTION

The present invention provides methods and compositions for treating the clinical manifestations of chronic or acute inflammation of the GIT, and/or of non-GIT sites in the body. The compositions include a source of polyunsaturated fatty acids, such as omega-3, and/or omega-6, and/or omega-9 fatty acids, including their triglycerides and esters; a drug used to treat the medical condition and optionally a source of pharmacologically active antioxidant (preferably a combination of the alpha- and gamma-isomers of tocopherol). Optionally also included are pharmaceutically acceptable carriers, including other essential and non-essential oils, diluents, viscosity-modifiers, stabilizers, erodable or swallowable matrices, and penetration enhancers.

### DETAILED DESCRIPTION OF THE INVENTION

The present invention provides an oral, enteral, or topical formulation comprising a source or form of polyunsaturated fatty acids, e.g. omega-3, and optionally, a source or form of pharmacologically active antioxidant such as tocopherols, and an anti-inflammatory non-steroid drug or anti-inflammatory steroid drug such as budesonide, for the treatment of inflammatory conditions of the GIT and/or of non-GIT sites in the body. The formulation combines the anti-inflammatory agent, e.g. budesonide, with an appropriate highly purified polyunsaturated fatty acid source, e.g., omega-3, or combination of highly purified or refined oils that can provide for a formulation containing fully solubilized drug or varying degrees of suspended drug. Changes in the amount of fatty acid enrichment, source of oil, type(s) of oil used (e.g., free fatty acid, ethyl ester, or triglyceride), and/or addition of pharmaceutically acceptable excipients, such as carriers, including other essential and non-essential oils, diluents, viscosity-modifiers, stabilizers, erodable or swallowable matrices, and penetration enhancers can be used to modify the solubility of the drug in the formulation. In addition, the same methods can be used to enhance or decrease the uptake of drug and/or oil components of the formulations, as well as modify various physical characteristics of the formulation such as viscosity, spread, and residence time. This invention specifically teaches that the delivery of drug, for example to the intestinal tract, can be controlled by the addition of appropriate excipients, as detailed above, or primarily by modifying the characteristics of the formulation using various types, or combinations of various types, of highly purified polyunsaturated fatty acids. The modification would be tailored to suit the chosen disease target, i.e., the type, location, and intensity of the inflammatory reaction present, as detailed in the examples. Preferably, but not exclusively, formulations are preferred that use highly refined oils that have inherent anti-inflammatory properties, e.g. the omega-3, omega-6, and omega-9 families of polyunsaturated fatty acids. The formulation thus effectively prevents or reduces acute flare-ups of certain such diseases; reduces the dose of drug required for effectiveness; and reduces the side effects associated with the drugs. The formulation also reduces the incidence or severity of the chronic systemic effects of the disease. Finally, the formulation reduces the incidence or severity of side effects of the drug component of the therapy. The action of the omega-3 and/or omega-6 and/or omega-9 and optionally antioxidant components, or the combination of all ingredients, may allow a lower initial dose, or final “tapered” dose, or maintenance dose of the drug component than would normally be prescribed and effective. This lessening of dosage of the drug, without compromising clinical efficacy, is part of the invention.

Surprisingly we have also found the presence of oils such as those enriched for omega-3 polyunsaturated fatty acids, quite apart from their potential therapeutic benefit, act as a carrier for the anti-inflammatory drug. In some instances the highly purified oils, e.g. omega-3, serve as a suspending medium for providing a slow drug release and in other cases they partially or fully solubilize the drug providing for faster drug release or drug uptake. The modification of the solubility of drug in the formulation is possible by addition of various agents, as detailed above, or the use of different types or combination of different types of highly purified oils.

If omega-3 oils are used, added benefits of the formulation include, firstly, the repletion of EFA, which have been shown to be lower than normal in some patients with inflammatory diseases of the intestine. Secondly, since the status of tocopherols (Vitamin E) is also often compromised under conditions of malnutrition or intestinal disease, an added benefit is the repletion of this vitamin with the formulation. Finally, tocopherols act as an antioxidant to protect polyunsaturated fatty acids.

The following terms used throughout this specification and in the appended claims are defined as follows: “treating” refers to any or all of the following:
prevention of the occurrence of a relapse, i.e., maintenance of remission
induction of remission of acute phases of GIT inflammation, regardless of etiology
either of the above in patients who are refractory to other medical treatments, and/or who are steroid-dependent or dependent on other medication
prevention or reduction of the effects of the disease, whether expressed in the GIT or elsewhere, i.e., systemically or locally
reduction of the required dose of drug whether in acute or non-acute phases
reduction of the side effects of the drug component of therapy

“Inflammation” refers here to redness, heat, swelling and pain in a local area of the body, often with pain or disturbed function, in reaction to an infection, or to a physical or chemical injury. Further, and relevant to this invention, biochemical mediators and effectors of inflammation include “eicosanoids”, which are produced by white cells from essential fatty acids, including omega-3. It is known that the eicosanoids produced from omega-3 are much less inflammatory, or anti-inflammatory, in their actions. These eicosanoids may also be “immuno-suppressive”. Eicosanoid production from omega-3 polyunsaturated fatty acids accounts, in part, for their anti-inflammatory properties. However, other physiological and biochemical properties of omega-3 polyunsaturated fatty acids could contribute to their salutory effects in inflammation. The anti-inflammatory properties of the omega-6 and omega-9 polyunsaturated fatty acids are much less well documented, but offer similar potential benefit through somewhat different but nevertheless overlapping mechanisms.

“GIT inflammation” refers here to the condition of inflammation occurring or threatening to occur in any portion of the GIT, from mouth to anus.

“Systemic or local inflammation” refers here to the condition of inflammation occurring or threatening to occur in any site of the body other than the GIT including the skin.

The drug or drugs present in the compositions and employed in the methods of this invention are anti-inflammatories as currently used in the treatment of inflammatory bowel diseases. Suitable anti-inflammatory drugs include corticosteroids such as prednisone, hydrocortisone, tixocortol, beclamethasone, budesonide; non-steroidal anti-inflammatory drugs (NSAIDs) including aminosalicylates such as 5-aminosalicylic acid (mesalamine) and its derivatives including sulfasalazine and olsalazine.

Other drugs that may be considered include nicotine and antibiotics such as metronidazole and ciprofloxacin.

“Omega-3” refers here to oils containing any or all polyunsaturated fatty acids of that chemical designation and their derivatives including triglycerides and esters, including but not limited to eicosapentaenoic acid (EPA) and docosahexaenoic acid (DHA). The fatty acids will be in the form of tri-, di-, or mono-glycerides, or in their “free” forms, such as ethyl esters. Sources include: animals (e.g., fish): plants (e.g., evening primrose, borage); other living sources, such as algae, bacteria, and yeast and their “bioengineered” derivative-forms; preparations that have been purified, modified or synthesized; and precursors of “omega-3”. Included as well will be derivatives that may be water-soluble. “Omega-6” and “Omega-9” refers here to oils containing any or all polyunsaturated fatty acids of that chemical designation and their derivatives as detailed for omega-3. The omega-3, omega-6, and omega-9 polyunsaturated fatty acids may typically be administered in amounts ranging from 1 to 6 grams per day, possibly up to 30 grams per day, but usually about 3 grams per day for most indications. However, since the exact amount of polyunsaturated fatty acids administered would be dependent upon the purity of the omega-3, omega-6, or omega-9 source, the site of drug administration, the relative amount of the anti-inflammatory drug used in combination with polyunsaturated fatty acids, and the indication being treated, the actual amount of polyunsaturated fatty acids used could be much lower than 1 g per day.

“Antioxidants” refers here to any naturally-occurring or synthetic chemicals which are known or believed to exert antioxidant effects in vitro and/or in vivo, that is pharmacologically active. Examples include several vitamins, such as tocopherols, ascorbate, and beta-carotene.

“Tocopherol” refers here to any source, form, isomer, or derivative of tocopherols (“vitamin E”), especially mixtures of the alpha- and gamma-isomers.

Vitamin E, which is usually D-alpha-tocopherol is available as a dietary supplement in 100, 400 and 1,000 IU capsules. For purposes of the present invention a usual daily dosage is about 400 IU. However, since the design of the invention is to be able to taper any one, or all of the components to facilitate intervention in the inflammatory process, the levels of antioxidant used may start from significantly lower than the daily dosage amount or may be absent. Furthermore, tocopherols, and other antioxidants, are frequently added to drug formulations specifically to decrease oxidation. Therefore, since our invention specifically pertains to a formulation containing omega-3, omega-6, or omega-9 polyunsaturated fatty acids, for the purposes of this invention the antioxidant levels will be limited only by the antioxidant activity of the antioxidant added to the formulation and/or the maximum tolerated dose.

The therapeutic procedures and compositions are here described with reference to humans, however they may also be employed in the field of veterinary medicine particularly in the treatment of domestic animals and commercial livestock.

For purposes of illustration, considering budesonide as the anti-inflammatory drug employed, dosages may vary up to 9 mg per day (recommended daily dose) in single or divided doses but are usually in the range of 3 mg per dose up to 3 doses per day. The maximum tolerated dose is unknown, and the maximum dose used would be dependent upon the comparison of efficacy and adverse manifestations by a medical professional. See also U.S. Pat. No. 5,643,602, the disclosure of which is hereby incorporated by reference, for a listing of illustrative anti-inflammatory steroids, dosages and other information.

The compositions of the invention include various presentations and formulations. For example all three “components” may be included in a single unit dosage form such as a capsule or as a liquid for oral ingestion or topical application for esophageal or stomach indications. Solid oral dosage forms will be formulated for delivery to the desired site of treatment such as by enteric coating tablets or capsules with a pH soluble-dependent coating. Topical and ophthalmic formulations, transdermal patches, solutions/suspensions/emulsions are also within the scope of the invention.

The essential components of the therapeutic combination may be administered separately at different times or simultaneously at the same time, sequentially or together conveniently in a single unit dosage.

The formulations of the present invention are appropriate for administration by any convenient route, including, but not limited to the following: oral, enteral (tube-fed), rectal, ophthalmic, dermatologic, subcutaneous, intramuscular, intraperitoneal, or intravenous.

Preferably the relative proportions of drug and omega-3 are such that the daily dose of omega-3 is from 0.1 to 30.0 grams per day and the relative proportions of the tocopherols and other active ingredients are such that the daily dose of tocopherols is from 1 to 1000 IU per day.

The components may be solubilized in an oil vehicle such as the omega-3 polyunsaturated fatty acids source.

The invention includes several embodiments, such as illustrated by but not limited to the following: In one embodiment, a composition is from a source of omega-3 and/or omega-6 and/or omega-9, a source of antioxidants, e.g., tocopherols, and a drug, e.g., budesonide.

In an embodiment the lipid component is fish (marine) oil, or fractions thereof which are relatively enriched in omega-3. A source of antioxidants such as tocopherols (e.g., alpha- and/or gamma-tocopherol) is added to the omega-3.

In an embodiment the lipid component is derived from a plant source, which is used for its high content of omega-3 or omega-6 or omega-9.

In an embodiment the lipid component is a synthesized, purified, or enriched source of the omega-3, omega-6, or omega-9 polyunsaturated fatty acids themselves (e.g., EPA, DHA for omega-3) which may be in the form of glycerides, methyl/ethyl esters, or other similar bioavailable forms.

In an embodiment, the inventive formulation contains, as active ingredients: a source of polyunsaturated acids (omega-3 and/or omega-6 and/or omega-9; a drug (e.g., budesonide); and an antioxidant such as tocopherols (e.g., alpha- and/or gamma-tocopherols). Other, similar drugs may also be employed, in place of or in conjunction with budesonide.

Since the invention is intended for treating inflammation of the GIT, as well as systemic and local inflammation multiple formulations are preferred, but not essential to the invention. Multiple formulations means a number of preparations which differ in the ratio of polyunsaturated acid to drug. For example, the ratio of omega-3 to antioxidant when present is preferred to be constant, as a function of stability considerations, although the ratio could be varied. This allows the tapering off of the drug component of the therapeutic combination, while maintaining a constant dose of nutrients (omega-3, tocopherols). Further, since patients respond differently, and the same patient may respond differently at different times, the “optimal” dose (i.e., ratio, omega-3:drug) may vary. This may initially have to be determined on a case-by-case basis. Multiple formulations allow selection of tailored dosing to accommodate this situation.

Another strategy for multiple formulations involves keeping the ratio, omega-3:drug constant, but reducing the total dose over time. Multiple, reduced dosing allows the patient to continue taking the same number of medications or unit dosage forms such as capsules or the like daily, in a “tapering” fashion. In addition, it should be noted that for formulations intended for release of formulation at specific intestinal targets, the type of oil used could be modified or chosen to provide an oil that would be rapidly, or less rapidly, absorbed, thereby providing drug for immediate, or delayed, uptake.

The present invention is further illustrated in the following general formulations related to the locus of the inflammatory condition to be treated.

Type I: Encapsulated fully solubilized budesonide in free fatty acids with high or low viscosity, enteric-coated for delivery to proximal small bowel.

Upon release: Immediate uptake of oil and budesonide with no effects on formulation spread.

Disease location: Systemic inflammation.

Type 2: Encapsulated fully suspended budesonide in triglyceride fatty acids with high or low viscosity, enteric-coated for delivery to proximal small bowel.

Upon release: Immediate uptake of oil and delayed uptake of budesonide, some formulation spread

Disease location: Systemic inflammation or proximal small bowel.

Type 3: Encapsulated fully solubilized budesonide in triglyceride mix with high or low viscosity, enteric-coated for delivery to distal small bowel.

Upon release: Some delay in uptake of oil, some immediate uptake of budesonide, formulation spreading, and minimal changes in residence time.

Disease location: Distal small bowel disease and ascending colon.

Type 4: Encapsulated fully suspended budesonide in triglyceride fatty acids with high viscosity, enteric-coated for delivery to distal small bowel.

Uptake: Some delay in uptake of oil, delay in uptake of budesonide, maximal formulation spread, and some increase in residence time.

Disease location: Distal small bowel, ascending and transverse.

Type 5: Encapsulated fully solubilized budesonide in free fatty acids with low viscosity, enteric-coated for delivery to ascending colon.

Uptake: Immediate uptake of oil and budesonide, formulation spread, and short residence time.

Disease location: Ascending and transverse colon.

Type 6: Encapsulated fully suspended budesonide in triglycerides with high viscosity enteric-coated for delivery to ascending colon.

Uptake: Delayed uptake of oil and budesonide, maximal formulation spread, and long residence time.

Disease location: Ascending and transverse colon.

It is understood that the application of the teachings of the present invention, to the conditions described, will be evident to one skilled in the art of preparing such formulations, and to one skilled in treating such medical conditions.

Additional features and advantages of the present invention are described below in preferred embodiments, which are intended as example, and not as limitation.

### Preferred Embodiments

### EXAMPLE #1

TABLE

| | Omega-3 | Tocopherol² | Budesonide A³ | Budesonide B³ |
|---|---|---|---|---|
| Dose: | (gm) | (IU) | (mg) | (mg) |
| Per | 1.0 | 100 | 2.25 | 1.5 |
| Capsule | | | | |
| Per four | 4.0 | 400 | 9.0 | 6.0 |
| Capsules | | | | |

¹Excipients
²Mixture of alpha- and gamma-isomers, ratio to be determined.
³Epimers as described in U.S. Pat. No. 5,643,602

The following contemplated clinical cases of Crohn's disease are presented as examples of methods of treatment for IBD, and not as limitations. Other illustrations of treating systemic or localized inflammatory reactions are not presented, since the principles involved are similar, and will be apparent to the skilled reader based upon review of an individual's clinical manifestation and the preferred embodiments of the inventive compositions.

### EXAMPLE #1

Two adults present in the emergency room of a hospital. Each is determined to be experiencing an acute relapse of Crohn's disease. Each has a history of CD over the past 5 years, with an acute episode about every 8-10 months. Patient #1 has CDAI score of 410, indicating a severe flare-up (CDAI is a commonly used index of disease severity; score value is directly related to severity). He is started on budesonide, 9 mg/day, twice daily. The dose is scheduled to be tapered off to 6, then 3 mg over the next 8-10 weeks, if the condition improves sufficiently.

Patient #2 has a CDAI score of 450, at least as severe as that of patient #1. This patient is also started on a formulation of 9 mg/day of budesonide, but formulated as the omega-3/tocopherol version (see Table). Tapering was also planned, over an 8 week period. Neither subject knew which formulation was being taken. Both subjects began consuming 100% of their nutrition from a simple medical food that was not “elemental” in its content, and contained small amounts of omega-3.

Patient #1 (budesonide) showed a gradual drop in CDAI, from 410 at baseline, to 290 at week 2; to 220 at week four; with gradual stabilization at 145 by 6 weeks. Budesonide was lowered to 6 mg/day at week 8; and to 3 mg/day: it was discontinued at week 12. The patient was able to consume a highly restricted diet, by 6 weeks. He graduated to “bland”, low residue foods, at week 10, and to “normal” foods at week 12. He was judged as fully recovered at week 14.

Patient #2 (budesonide+omega-3) showed a drop in CDAI from 450 at baseline, to 220 at week two; and a further drop to 170 at week 4; stabilizing at 120 by week 6. This patient began consuming a “bland”, low residue diet by week 6, and normal foods by week 8-9. He was judged fully recovered at week 10.

### EXAMPLE II

Two patients with CD, in remission for the last 10 months, were screened and found to be at high risk of relapse. This was judged on the basis of elevated biochemical markers of inflammation, namely, C-Reactive Protein (CRP), alpha-1 acid glycoprotein, and erythrocyte sedimentation rate (ESR). Patient #1 began receiving a “prophylactic” dose of budesonide, 2 mg/day, BID. Patient #2 began receiving a formulation of budesonide in a daily dose of 1.0 mg/day; omega-3, in a dose of 3.0 grams/day; and tocopherol in a dose of 75 IU/day. Both patients also happened to manifest extra-intestinal (systemic) symptoms, including arthritis and conjunctivitis. Neither patient, nor physician, knew the identity of their prescription. Each formulation was intended to be continued until a relapse occurred, or for one year.

Patient #1 (budesonide) showed increases in the values of the inflammatory markers at month 2, a greater increase at month 4. Shortly after the 4-month blood sample was analyzed, the patient began experiencing worsening clinical symptoms of GIT inflammation, and was judged to be in full relapse before month 5 was reached. She was admitted to hospital for stabilization, and began receiving the preparation containing budesonide+omega-3; and went into remission by week 8 of this treatment. Patient's systemic symptoms did not change.

Patient #2 showed stable biochemical risk factors at month 2, a noticeable drop at month 4, and fully normal values by month 6. The patient had not relapsed when the study treatment was terminated at month 12. Subject's systemic symptoms were significantly improved in less than 6 months. Subject was weaned from budesonide at this time, but elected to continue taking commercial preparations of omega-3 thereafter.

1. A pharmaceutical composition for the treatment of an inflammatory condition in a mammal consisting essentially of budesonide or a prodrug or derivative thereof, in a highly purified omega-3 and/or omega-6 and/or omega-9 polyunsaturated fatty acids, or derivatives thereof, optionally also including a pharmaceutically acceptable carrier, a diluent, a viscosity modifier, a stabilizer, an erodable or swallowable matrix, and a penetration enhancer.

2. The pharmaceutical composition of claim 1, wherein the antioxidant is a tocopherol.

3. The pharmaceutical composition of claim 2, wherein the tocopherol is a mixture of alpha- and gamma-isomers.

4. The pharmaceutical composition of claim 1, where the omega-3 and/or omega-6 and omega-9 polyunsaturated fatty acid, or a prodrug or a derivative thereof, are in the form of a tri-, di, or mono-glyceride, methyl/ethyl ester, free fatty acid, or other bio-available form.

5. The pharmaceutical composition of claim 4, wherein the budesonide, or a prodrug or derivative thereof is fully solubilized.

6. The pharmaceutical composition of claim 4, wherein the budesonide, or a prodrug or derivative thereof is fully suspended.

7. The pharmaceutical composition of claim 4, wherein the budesonide, or a prodrug or derivative thereof is partially suspended.

8. The pharmaceutical composition of claim 1, wherein the composition provides from 0.1 to 30 grams per day of omega-3 and/or omega-6 and/or omega-9 polyunsaturated fatty acids, or derivatives thereof.

9. An orally or enterally administrable composition for the treatment of an inflammatory conditions of the gastrointestinal tract, consisting essentially of an effective amount of budesonide, or a prodrug or derivative thereof, highly purified omega-3 and/or omega-6 and/or omega-9 polyunsaturated fatty acid; and an antioxidant, optionally including a pharmaceutically acceptable carrier, a diluent, a viscosity-modifier, a stabilizer, and an erodable or swallowable matrix.

10. An orally administrable composition for the treatment of a systemic or local inflammatory condition consisting essentially of an effective amount of budesonide, or a prodrug or derivative thereof, highly purified omega-3 and/or omega-6 and/or omega-9 polyunsaturated fatty acid, and an antioxidant, optionally including a pharmaceutically acceptable carrier, a diluent, a viscosity-modifier, a stabilizer, an erodable or swallowable matrices, and/or a penetration enhancer.

11. The pharmaceutical composition of claim 9, wherein the composition is encapsulated and enteric-coated for release and/or delivery of drug of about 55% into the proximal small bowel.

12. The pharmaceutical composition of claim 9, wherein the composition is encapsulated and enteric-coated for release and/or delivery of drug of about 25% into the proximal small bowel.

13. The pharmaceutical composition of claim 9, wherein the composition is encapsulated and enteric-coated for release and/or delivery of drug of about 25% into the ascending and transverse colon.

14. A topically administrable composition for the treatment of a systemic or local inflammatory condition consisting essentially of an effective amounts of budesonide, or a prodrug or derivative thereof; highly purified omega-3 and/or omega-6 and/or omega-9 polyunsaturated fatty acid; and an antioxidant; optionally including a pharmaceutically acceptable carrier, a diluent, a viscosity-modifier, a stabilizer, an erodable matrix, and/or a penetration enhancer.

15. A pharmaceutical composition for the treatment of an inflammatory condition in a mammal, the composition consisting essentially of a steroid or non-steroidal anti-inflammatory drug; highly purified omega-3 and/or omega-6 and/or omega-9-polyunsaturated acid or a derivative thereof, and optionally including a pharmaceutically acceptable carrier, a diluent, a viscosity-modifier, a stabilizer, an erodable and swallowable matrix, and/or a penetration enhancer.

16. A pharmaceutical composition for the treatment of an inflammatory condition in a mammal consisting of budesonide or a prodrug or derivative thereof, in a highly purified omega-3 and/or omega-6 and/or omega-9 polyunsaturated fatty acids, or derivatives thereof, optionally also including a pharmaceutically acceptable carrier, a diluent, a viscosity modifier, a stabilizer, an erodable or swallowable matrix, and a penetration enhancer.

17. An orally or enterally administrable composition for the treatment of an inflammatory conditions of the gastrointestinal tract, consisting of an effective amount of budesonide, or a prodrug or derivative thereof, highly purified omega-3 and/or omega-6 and/or omega-9 polyunsaturated fatty acid; and an antioxidant, optionally including a pharmaceutically acceptable carrier, a diluent, a viscosity-modifier, a stabilizer, and an erodable or swallowable matrix.

18. An orally administrable composition for the treatment of a systemic or local inflammatory condition consisting of an effective amount of budesonide, or a prodrug or derivative thereof, highly purified omega-3 and/or omega-6 and/or omega-9 polyunsaturated fatty acid, and an antioxidant, optionally including a pharmaceutically acceptable carrier, a diluent, a viscosity-modifier, a stabilizer, an erodable or swallowable matrices, and/or a penetration enhancer.

19. A topically administrable composition for the treatment of a systemic or local inflammatory condition consisting of an effective amount of budesonide, or a prodrug or derivative thereof, highly purified omega-3 and/or omega-6 and/or omega-9 polyunsaturated fatty acid; and an antioxidant; optionally including a pharmaceutically acceptable carrier, a diluent, a viscosity-modifier, a stabilizer, an erodable matrix, and/or a penetration enhancer.

20. A pharmaceutical composition for the treatment of an inflammatory condition in a mammal, the composition consisting of a steroid or non-steroidal anti-inflammatory drug; highly purified omega-3 and/or omega-6 and/or omega-9-polyunsaturated acid or a derivative thereof, and optionally including a pharmaceutically acceptable carrier, a diluent, a viscosity-modifier, a stabilizer, an erodable and swallowable matrix, and/or a penetration enhancer.
